(19) United States
(12) Patent Application Publication
(10) Pub. No.: US 2021/0169156 A1
(43) Pub. Date: Jun. 10, 2021

(54) GARMENT WITH SELECTIVE SUPPORT AND COMPRESSION

(71) Applicant: Bittyrina LLC, Sherman Oaks, CA (US)

(72) Inventor: Brett Lindsay Murphy, Sherman Oaks, CA (US)

(21) Appl. No.: 17/096,818

(22) Filed: Nov. 12, 2020

Related U.S. Application Data
(60) Provisional application No. 62/934,199, filed on Nov. 12, 2019.

(51) Int. Cl. A41D 13/00; A41D 31/30; A61F 13/08; A41C 3/08

(52) U.S. Cl. CPC A41D 13/0017; A41D 31/30; A41D 2300/322; A41C 3/08; A61F 13/08

(57) ABSTRACT

A garment with selective compression may include multiple regions associated with different parts of a user's body, such as the arms, chest, midsection, buttocks, and legs. Each region of the garment may have a different level of compression. Higher levels of compression may provide support and alter the shape of the associated body part, while lower levels of compression may provide a smooth appearance without altering shape. By having selective compression levels for each region, the garment can smooth or shape different parts of the user's body in order to achieve a user's desired appearance, in addition to selectively providing different levels of support and increasing overall comfort and functionality.

### CROSS-REFERENCE TO RELATED APPLICATIONS

This application claims priority to U.S. Provisional Application No. 62/934,199, titled “GARMENT WITH SELECTIVE SUPPORT AND COMPRESSION,” and filed Nov. 12, 2019, upon which is incorporated by reference hereto.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### TECHNICAL FIELD

The present disclosure relates generally to garments, and more specifically, to a garment that selectively provides regions providing varying levels of compression and support.

### BACKGROUND

Users may wear specific garments designed to provide support to various regions of the body. For example, a compression shirt may provide compression to the torso and arms of a user. As another example, a compressive undergarment may compress the waist and midsection of the user.

Compressive garments may be worn by the user to generally smooth and reduce the appearance of various portions of the body. For example, a compressive undergarment may reduce the appearance of cellulite on the user.

In many cases, users may generally desire to wear a garment that provides compression and support to various regions of the body while providing minimal compression in other areas. For example, a user may want greater compression and support to their midsection and less compression in the buttocks area of the user. However, many compressive garments may only provide uniform compression across the entire garment, preventing selective compression to various regions of the body.

### BRIEF DESCRIPTION OF THE DRAWINGS

The present embodiments are illustrated by way of example and are not intended to be limited by the figures of the accompanying drawings.

FIG. 1A illustrates a front view of a garment with selective compressive regions.

FIG. 1B illustrates a back view of a garment with selective compressive regions.

FIG. 2 illustrates a garment with selective compressive regions as worn by a user.

FIG. 3 illustrates a garment with selective compressive regions as worn by a user engaging in exercise.

FIG. 4 illustrates an interior view of a garment with selective compressive regions.

FIG. 5 illustrates a garment with selective compressive regions including a front fastener.

FIG. 6 illustrates a garment with selective compressive regions including a back fastener.

Like reference numerals refer to corresponding parts throughout the figures and specification.

### DETAILED DESCRIPTION

Users may wear specific garments designed to provide support to various regions of the body. Compressive garments may be worn by the user to generally smooth and reduce the appearance of various portions of the body. For example, a compressive undergarment may reduce the appearance of cellulite on the user.

In many cases, users may generally desire to wear a garment that provides compression and support to various regions of the body while providing minimal compression in other areas. This may smooth out specific regions of the body while accentuating other regions of the body, increasing aesthetics and a desired appearance of the user. As an example, a user may want greater compression and support to their midsection and less compression in the buttocks area of the user.

However, many compressive garments may only provide uniform compression across the entire garment, preventing selective compression to various regions of the body. For example, a compressive bodysuit may provide uniform compression across the body of the user. Such a garment may be uncomfortable to the user and lower overall desired aesthetics of the user, as specific regions are unable to be accentuated by the user.

The present embodiments relate to a garment configured to provide varying levels of compression and support selectively across a body of a user. The garment may selectively provide greater compression to some regions, such as a midsection region of the user, while providing less compression in other regions, such as a buttocks region.

Selectively providing varying levels of compression and support across the garment may provide a desired aesthetic for a user, as the garment can compress and smooth out specific parts of the body while accentuating other parts of the body. The garment can provide greater comfort while wearing the garment and may minimize the appearance of moving and shaking, smooth dips and dimples, and accentuate natural curves of the body of the user.

The garment may include built-in bra support, shapewear, and selective compression to assist in supporting various body parts. The fabric may include an anti-microbial material to mitigate proliferation of microorganisms and allow the user to wear the garment during many occasions. For example, the user may work from home, engage in a workout, then eat dinner, all while wearing the garment. The garment may be worn as either an undergarment or as an outfit. The compression may provide comfortable firming in select areas. For example, the selective compression may eliminate the appearance of cellulite in thighs, while only smoothing the buttocks of the user.

Further, the garment may include any of a waist-cinching zipper, gentle and effective arm compression, and built-in shapewear. The garment may include a light-weight lining of the bodice, waist, buttocks, and sleeves that can allow for the insertion of bra cups and additional compression mechanisms (padding, lipo foam, binding, etc.). Unlike other garments (e.g., shapewear and post-surgical garments), the garment as described herein is not solely designed to be hidden under clothing. It is a single garment that is multi-dimensional and operates in many capacities. For example, the garment may be used for fashion, as activewear, for maternity or post-partum wear, or for post-surgery use.

The garment may also include any of varying pant and sleeve lengths. A fastener, such as a zipper, may be placed down the front, down the back, or on the side along the waist. Heavy compression may be included in the arm sleeves, the thigh area, or the stomach and waist area. A lighter compression may be included in the buttocks area.

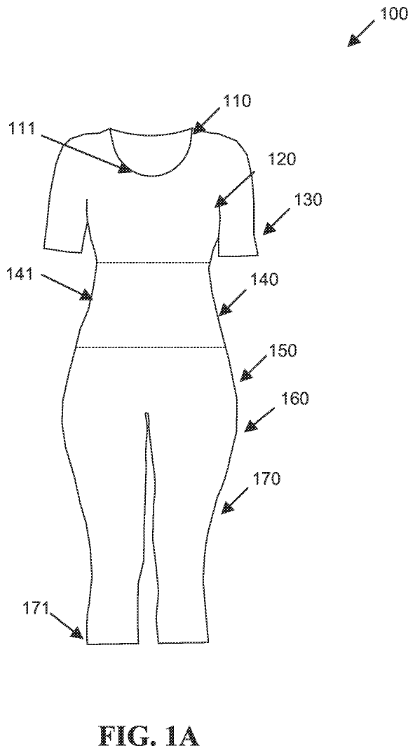

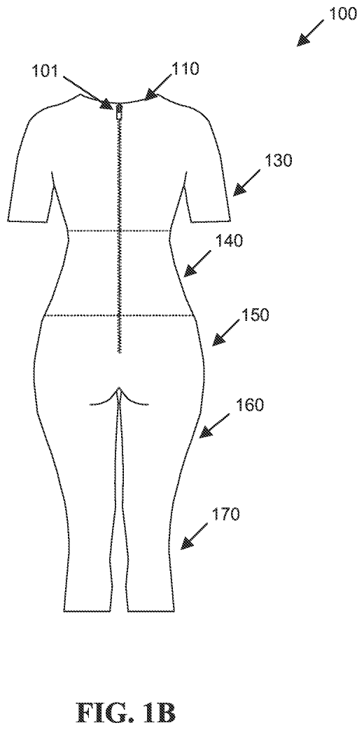

FIG. 1A illustrates a front view of a garment 100 with selective compressive regions. In the embodiment as shown in FIG. 1A, the garment can include a body suit with multiple regions configured to fit between the neck and ankles of the user. In some embodiments, the garment as described herein can be configured to fit around any region of the body, including as a shirt, waistband, or pair of pants.

As shown in FIG. 1A, the garment 100 can include multiple regions. Example regions for the garment can include a neck region 110 near the neck of the user, a chest region 120 around the chest of the user, an arm region 130 around the arms of the user, a midsection region 140 around a midsection of the user, a buttocks region 150 around a buttocks of the user, a thigh region 160 around the thighs of the user, or a leg region 170 around the legs of the user.

The neck region can include a plunging neckline 111 that provides support such that the chest of the user is lifted and enhanced. The neck region can also include a wide neckline that makes it easier for the user to pull the garment up or down, for example when putting on or removing the garment. The arm region can include compression sleeves that can make arms appear toned and trim. The compression sleeves can also assist in holding the garment more securely on the user. Shapewear 141 built into midsection region of the garment may compress bulges or cellulite to provide a desired aesthetic. For example, the user may desire to have an appearance including a slimmer waist and toned arms but keep a more natural appearance in the legs and buttocks. In such an example, the user can wear a garment including higher compression in the midsection and arms regions, but lesser compression in the legs and buttocks regions.

The garment may include any suitable fabric. The garment may include an antimicrobial activewear fabric, such as polyester, polyester-vinyl composites, vinyl, acrylics, synthetic fibers (e.g., elastane), or a combination of any such fabrics. The fabric(s) of the garment may be flexible such that the garment provides compression as described herein. In some embodiments, the garment may include a waterproof fabric such that the garment can be worn while the user swims. Example waterproof fabrics include PUL fabric, TPU fabric, and polyester or nylon treated with water repellent coatings.

The leg region 170 of the garment may lightly compress legs and minimize, for example, appearance of any cellulite. The leg region may include varying lengths. For example, the leg region can extend between any of the thigh region 160 of the user and an ankle region of the user. In other words, the leg region may comprise a shorts length, a full pant length, a mid-ankle length, a calf length, or any other suitable length. FIG. 1A shows an embodiment with a calf length leg region 171.

As noted above, the regions of the garment can have varying levels or degrees of compression. A level of compression may be represented by a measurement of pressure, such as a millimeter of mercury (mmHg). A higher level of compression will be represented by a higher pressure in mmHg and alter the appearance of the body of the user from a natural state to a greater degree than a lower level of compression. For example, a minimal level of compression may have pressure <8 mmHg and provide a smoothing effect on the body of the user without affecting the size or shape of the body. A mild compression may range between 8 to 15 mmHg and provide a greater smoothing effect on the body of the user and adjust the shape and size of the body while providing light support. A moderate compression may range between 15 to 20 mmHg and adjust the apparent shape of the user's body to a greater degree than a mild compression along with providing greater support. A firm compression may range between 20 to 30 mmHg, and may be used to change the appearance of the user's body to a greater degree than mild or moderate compressions. A firm compression may also provide more support than mild or moderate compressions. As an example, a user may desire to shrink the user's waist dramatically, tone and firm the user's thighs, and smooth other areas of the user's body without reducing the apparent size or altering the shape of those areas. Accordingly, a midsection region 140 can include a firm level of compression, a thigh region 160 can include a moderate level of compression less than that of the midsection region, and the arm region 130 or leg region 170 can include a mild level of compression less than that of the thigh region. In this example, other regions, such as the buttocks region and the chest, can have a minimal level of compression.

The level of compression needed to achieve a desired aesthetic may vary with the user of the garment. For example, a user with a 34 inch waist size may use a higher level of compression in the midsection region than a user with a 32 inch waist size to achieve a desired waist size. In another example, a user with more elastic skin may use a lower level of compression than a user with less elastic skin to achieve a desired smooth appearance.

In some embodiments, the garment may include one or more pockets. The pockets can include suitable sizes to receive various items such as a credit card, mobile phone, or key ring. The pockets may be disposed on any region of the garment. For example, the pockets may be disposed on the outer thigh such that the user can easily access the pockets. A pocket may also be disposed on a sleeve to hold a phone while the user exercises.

FIG. 1B illustrates a back view of a garment 100 with selective compressive regions. As shown in FIG. 1B, the garment may include a fastener 101 that allows for the user to crunch the midsection and improve the silhouette of the user. Example fasteners can include a zipper, buttons, string, or another suitable fastener. The fastener may allow for compression of the garment to the body of the user. In FIG. 1B, fastener 101 is shown as a zipper, but other suitable fasteners may be used.

As noted above, the buttocks region can include a lightest compression to create a smooth appearance but not shrink the size of the buttocks of the user. For example, a lowest level compression of 0-8 mmHg may create a smoother appearance without shrinking the size of the buttocks for some users. Other users may use a mild level of compression of 8-15 mmHg to further enhance smoothing while only slightly shrinking the size of the buttocks.

In one embodiment, a garment may comprise multiple regions. For example, a midsection region 140 can include a firm level of compression. An arm region 130, leg region 170, or thigh region 160 can include a second level of compression less than that of the midsection region. A buttocks region 150 or a chest region can include a mild or lower level of compression. The varying level of compression can provide greater comfort and selective support to various parts of a body of a user. The garment in the previous example may have more support in the arms, legs, and thighs, while having less support in the buttocks and chest. In another example, a user who wants more support in the chest may wear a garment with a higher level of compression, such as a moderate compression in the 15-20 mmHg range.

The garment may include a zipper 101 that can engage and disengage the compression between the garment and the body of the user. The compression between the garment and the body of the user may also be affected by adjusting the length and placement of the zipper. For example, the zipper 101 as shown in FIG. 1B is placed on the back of the garment and extends from below the midsection region 140 to the neck region 110, which allows for greater compression of the midsection and waist. In addition, the shape of the zipper may be used to enhance the appearance of the user. For example, FIG. 1B shows a straight zipper 101, but adding a curved portion to the bottom of the zipper may create the appearance of a more shapely buttocks.

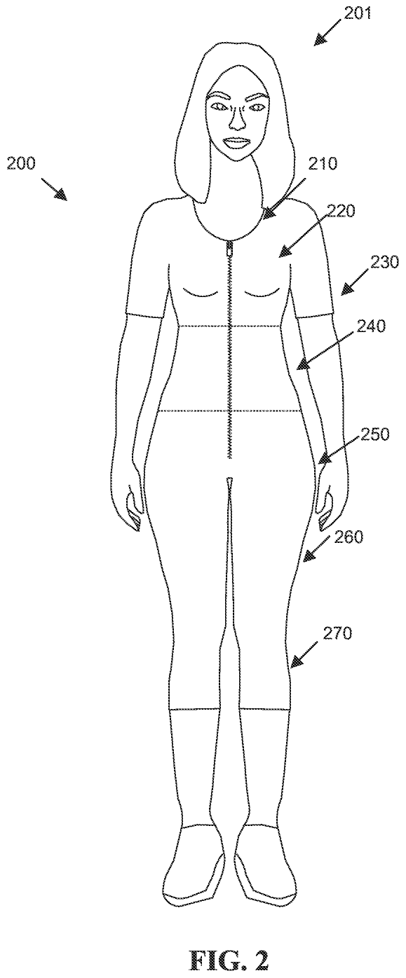

FIG. 2 illustrates a garment 200 with selective compression regions as worn by a user 201. In the embodiment as shown in FIG. 2, the garment can include multiple regions, such as a neck region 210, a chest region 220, an arms region 230, a midsection region 240, a buttocks region 250, a thigh region 260, and a legs region 270.

The legs region 270 may include varying lengths, such as shorts length, calf length, ankle length, or full pant length. In the embodiment as shown in FIG. 2, the legs region 270 is shown with a calf length. The legs region may include varying levels of compression. For example, the legs region may include a mild level of compression to smooth appearance and minimize cellulite. In other embodiments, the legs region may include a higher level of compression, such as moderate compression, to provide a firm appearance, provide support, or increase blood flow during exercise.

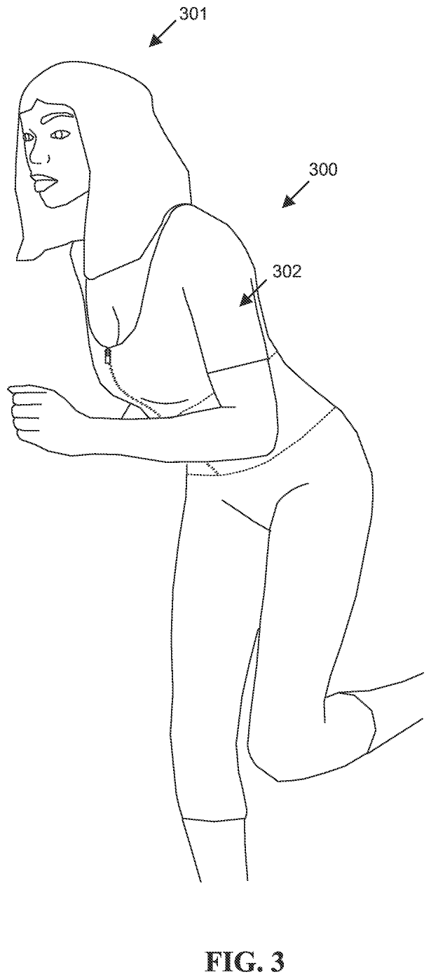

FIG. 3 illustrates a garment 300 with selective compression regions as worn by a user 301 engaging in exercise. The garment may include an antimicrobial fabric 302 to minimize the growth of bacteria and viruses, as well as minimize odors. Example antimicrobial fabrics may be the result of treatment with copper, zinc, or silver based compounds, such as Microban® and Silpure®. The garment is not limited to aerobic exercise as shown in FIG. 3, but may also be used for weight training, yoga, or other exercises. In some embodiments, the garment may include a waterproof fabric such that the user can swim while wearing the garment. Furthermore, the garment is suitable for, but not limited to, exercise activities. The garment may be used in a variety of circumstances, including relaxing at home, running errands outside, or being worn as a base layer underneath other clothing. In some instances, the user benefits from a garment with selective compression by enhancing the user's desired aesthetics as described herein. The user may then wear the garment by itself, or the user may use the enhanced aesthetics in conjunction with other clothing. For example, the user may use a garment with selective compression to cinch the user's waist, which then enables the user to more easily fit into a pair of pants. In another example, the user may wear the garment without layering other clothing, as shown in FIG. 3.

In addition, selective compression may increase the comfort of the garment, enabling the user to wear the garment all day, unlike garments without selective compression that may feel too tight in some areas of the user's body while being too loose in other areas. An antimicrobial fabric may further enable the user to wear the garment for longer periods of time. For exercise, compression may benefit the user by providing support, reducing fatigue, increasing blood flow, and decreasing recovery time.

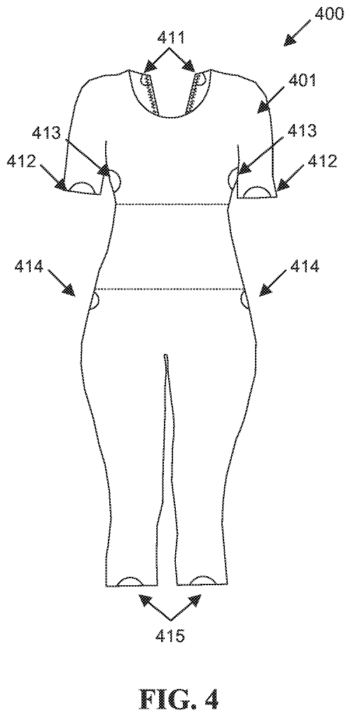

FIG. 4 illustrates an interior view of a garment 400 with selective compressive regions. In some embodiments, the garment may include an interior lining 401. The interior lining can include slots located in different regions of the garment, such as slots 411 in the back of the garment, slots 412 in an arm region, slots 413 in a chest region, slots 414 in an upper leg region, and slots 415 in a lower leg region of the garment. The slots can be used to hold various items, such as hot or cold packs, bra cups, or additional compression mechanisms such as padding, binding or lipo foam. For example, a user suffering from shoulder pain may choose to insert a cold pack into a slots 411 in the back of the garment 400. In another example, the user may insert bra cups into slots 413 in the chest region to provide additional support for the bust. These slots may be located anywhere on the garment and are not limited to those locations as shown in FIG. 4. For example, slots may be located in a knee region, suitable for knee pads, or in a midsection region. Similarly, slots may be located on the exterior of the garment, for example to create an exterior pocket suitable for cash or a cell phone. As illustrated in FIG. 4, slots 414 in an upper leg region are included in the interior lining 401 to create an interior pocket. Slots instead placed in the exterior could be used to mimic a traditional pants pocket placement, for example to hold cards, money, or a cell phone.

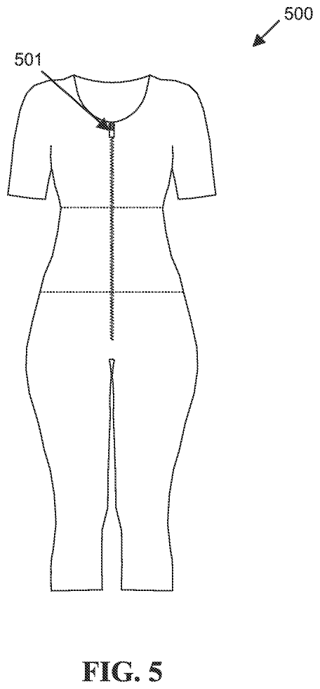

FIG. 5 shows an example garment 500 including a zipper 501. The zipper 501 can extend along a central portion of the garment from a lower midsection area to an upper midsection area. The zipper 501 in FIG. 5 extends beyond the midsection to a neck area of the garment. In some embodiments, a zipper placed on the front of the garment in this manner can accentuate the hips, add curvature to the waist, and lift the bust of the user. In addition, the shape of the zipper can affect the appearance of the user. For example, a curved portion can be included in the zipper 501 near the chest to create the appearance of a fuller chest.

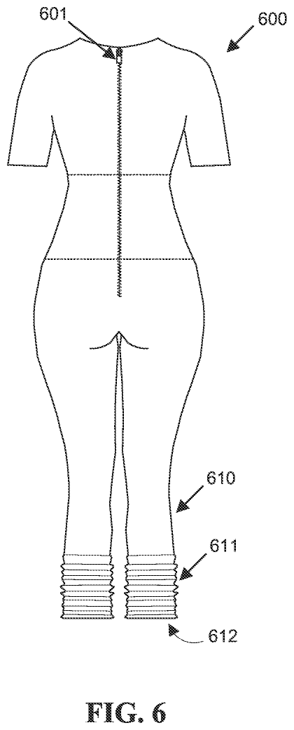

FIG. 6 illustrates an example garment 600 including a zipper 601 on the back of the garment. The zipper can extend along a central portion of the garment from a lower midsection area to an upper midsection area. The zipper 601, as shown in FIG. 6, extends beyond the midsection to a neck area of the garment. In certain embodiments, the zipper can be placed on the back of the garment to enhance the appearance of the waist and buttocks. As noted above, the shape of the zipper can affect the appearance of the user. For example, a curved portion can be included near the bottom of the zipper to give the appearance of a more shapely buttocks.

The garment may include a legs region 610 of varying lengths. As shown in the embodiment in FIG. 6, the legs region 610 may include a full pant length. The legs region may further include a ruched portion 611. In some embodiments, the ruched portion may enable the user to adjust the length of the legs region to their liking. In addition, the ruched portion may further include a sock portion 612 so the user can pull the sock portion over the user's feet, removing the need to wear a separate pair of socks

From the foregoing, it will be appreciated that specific embodiments of the invention have been described herein for purposes of illustration, but that various modifications may be made without deviating from the scope of the invention. Accordingly, the invention is not limited except as by the appended claims.

What is claimed is:

1. A garment that includes multiple portions, each having a level of compression, the garment comprising:
a first portion extending from a neck area of the garment to an upper portion of a midsection area of the garment, the first portion having a first compression level;
a second portion extending from the upper portion of the midsection area to a lower portion of the midsection area and having a second compression level that is greater than that of the first compression level; and
a third portion extending from the lower portion of the midsection area to a leg area of the garment, the third portion having a third compression level.

2. The garment of claim 1, wherein the level of compression is indicative of a force per unit area exerted by the garment on an associated region of a body of a wearer.

3. The garment of claim 1, wherein the second compression level is between 20-30 mmHg.

4. The garment of claim 1, the third compression level having a lesser compression than that of the second compression level.

5. The garment of claim 1, wherein the garment includes an antimicrobial fabric.

6. The garment of claim 1, further comprising:
a zipper extending along a central portion of the garment from the lower midsection area to the upper midsection area.

7. The garment of claim 1, wherein the second portion is stitched into the first portion and the third portion to comprise a unitary garment, wherein the second compression level of the second portion is configured to compress a midsection of a wearer.

8. The garment of claim 1, further comprising:
a bra-support built into the first portion.

9. The garment of claim 1, wherein the first portion includes sleeves configured to extend along an arm portion of a wearer.

10. The garment of claim 7, wherein the sleeves include a sleeve compression level, the sleeve compression level having a lesser compression than that of the second compression level.

11. The garment of claim 1, further comprising:
an interior lining, wherein the interior lining includes a slot.

12. The garment of claim 1, wherein the third portion includes a ruched portion, the ruched portion configured to extend to a foot of a wearer when stretched.

13. A garment comprising:
a first portion extending from a neck region of the garment to an upper portion of a midsection area of the garment, the first portion including a first compression level;
a second portion extending from the upper portion of the midsection area to a lower portion of the midsection area and having a second compression level that is greater than that of the first compression level; and
a third portion extending from the lower midsection area to a leg area of the garment, the third portion having the first compression level, wherein the garment includes an antimicrobial fabric; and
a zipper extending along a central portion of the garment from the lower midsection area to the upper midsection area.

14. The garment of claim 13, wherein the second portion is stitched into the first portion and the second portion to comprise a unitary garment, wherein the second compression level of the second portion is configured to compress a midsection of a wearer.

15. The garment of claim 13, further comprising:
a bra-support built into the first portion.

16. The garment of claim 13, wherein the first portion includes sleeves configured to extend along an arm portion of a wearer.

17. A method for manufacturing a garment that includes varying levels of compression, comprising:
affixing a first portion extending from a neck region of the garment to an upper portion of a midsection area of the garment to a second portion extending from the upper portion of the midsection area to a lower portion of the midsection area, the first portion having a first compression level and the second portion having a second compression level that is greater than that of the first compression level; and
affixing the second portion to a third portion extending from the lower midsection area to a leg area of the garment, the third portion having the first compression level.

18. The method of claim 17, further comprising:
affixing a zipper extending along a central portion of the garment from the lower midsection area to the upper midsection area.

19. The method of claim 17, wherein the second compression level of the second portion is configured to compress a midsection of a wearer.

20. The method of claim 17, wherein the first portion includes sleeves configured to extend along an arm portion of a wearer.
